(19) United States
(12) Patent Application Publication
(10) Pub. No.: US 2007/0112326 A1
(43) Pub. Date: May 17, 2007

(54) Device for Automated Delivery of a Liquid Medicament into a Patient's Body

(76) Inventors: David Bosshard, Wengi (CH); Axel Remde, Lutzelfluh-Goldbach (CH); Gilbert Schiltges, Kirchberg (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/555,776

(22) Filed: Nov. 2, 2006

(30) Foreign Application Priority Data
Nov. 4, 2005 (EP) 05 024 057.1

(51) Int. Cl. A61M 31/00

(52) U.S. Cl. 604/500; 604/132

(57) ABSTRACT

A device for automated subcutaneous delivery of insulin into a patient's body, wherein the device includes an infusion cannula for subcutaneous introduction of the insulin into the body, an insulin reservoir having an amount of insulin which is non-fatal for a person on short-duration subcutaneous administration, and an automated conveying device for conveying the insulin from the reservoir to the infusion cannula which, after manual actuation by the patient, starts automatically conveying the insulin, after a delay of from four to eight hours, and conveys the complete amount of insulin in the reservoir to the infusion cannula within a period not exceeding four hours. Methods of making and using the device are encompassed.

### CROSS-REFERENCE TO RELATED APPLICATION(S)

This application claims priority to European Patent Application No. 05 024 057.1, filed on Nov. 4, 2005, the content of which is incorporated in its entirety by reference herein.

### BACKGROUND

The present invention relates to devices for delivering, injecting, administering, dispensing or infusing substances, and to methods of making and using such devices. More particularly, it relates to a method for automated delivery of a liquid medicament to an infusion cannula, to an application or use of the method, to a device for automated delivery of a liquid medicament into a patient's body, to making and using the device, to a disposable pack of insulin as a reservoir for the device, and to a disposable pack of a physiological solvent to form a reservoir for the device.

Automated delivery of liquid medicaments into the body may be used in the case of patients who have a continuous requirement, which may vary over the course of the day, for a medicament or therapeutic substance, including those which can be administered exclusively subcutaneously. Examples of specific applications are certain pain therapies and the treatment of diabetes, for which computer-controlled medicament pumps which can be worn on the body are employed. Such pumps may comprise an amount of medicament sufficient for several days in a reservoir and convey the liquid medicament in accordance with a preprogrammed daily profile from the reservoir through an infusion cannula into the patient's body. However, such medicament pumps are relatively costly and require appropriate safety functions, not least because of the fact that they may hold a potentially fatal dose of medicament. Further, their use may require special knowledge and a certain skill in handling electronic instruments, so that their use is restricted to certain groups of patients.

A problem which emerges especially in insulin therapy for patients with type I and type II diabetes who do not use a computer-controlled insulin pump, but inject the insulin manually several times a day, is that typically there is a very great need for insulin in the early hours of the morning (“dawn phenomenon”), that is to say at a time when the patient is normally asleep. If no special measures are taken, this leads to blood glucose levels which are in some cases very high in the morning and to a correspondingly unsatisfactory overall control with all its known negative consequences.

The dawn phenomenon is currently treated according to the state of the art. Besides with the aforementioned insulin pump therapy which can be achieved for only a limited group of patients, it also may be treated by the possible injection in the evening of an insulin with a delaying profile of action so that an adequate amount of insulin is available in the morning. However, because of the limited possibilities of achieving the profiles of action, this frequently does not lead to satisfactory results. In addition, the onset of the effect may, because of variations in absorption, take place either too early or too late. Another possible solution is to wake up or be awakened at a suitable time during the night (e.g. 4 a.m.) and to inject a suitable amount of insulin manually. However, this is associated with considerable personal effort and a corresponding loss of quality of life.

### SUMMARY

An object of the present invention is to provide methods and devices which do not exhibit the disadvantages of the prior art or at least partly avoid or alleviate them.

In one embodiment, the present invention comprises a device for automated delivery of a substance into a patient's body, comprising a cannula for introduction of the substance into the body, a reservoir containing the substance, and a conveying device for conveying the substance from the reservoir to the cannula, wherein, after actuation and a delay, the conveying device automatically starts conveying the substance and, after the start of conveying, conveys substantially all the substance to the cannula within a selected time.

Accordingly, in one embodiment, the present invention comprises a method for automated delivery of a liquid medicament to an infusion cannula, which may be designed as infusion needle. In this case, in a first step, the medicament to be administered is provided in a reservoir in an amount which is non-fatal for a person on short-duration (within a few seconds) subcutaneous administration. Then an automated conveying device is actuated, in some preferred embodiments manually, and starts, with a delay of from four to eight hours, to convey the total amount of medicament provided from the reservoir to the infusion cannula within not more than four hours.

A second aspect of the present invention relates to a device for automated delivery of a liquid medicament into a patient's body which, in one preferred embodiment, is suitable for carrying out an embodiment of the method according to the present invention. The device comprises an infusion cannula for subcutaneous introduction of the medicament into the patient's body, which may be designed as infusion needle. It additionally comprises a reservoir which comprises an amount, which is non-fatal for a person on short-duration (within a few seconds) subcutaneous administration, of the liquid medicament to be delivered and/or of a physiological solvent for a liquid medicament. The device further comprises an automated conveying device for conveying the liquid medicament and/or the physiological solvent from the reservoir to the infusion cannula, which is designed such that it automatically starts conveying, after, in some embodiments, a manual actuation and with a delay of from four to eight hours, and conveys the total amount of fluid provided in the reservoir to the infusion cannula within a period not exceeding four hours after the start of conveying.

An advantage of the present invention over the prior art is that the reservoir does not contain a potentially fatal amount of active ingredient and is virtually completely emptied at each application, so that extensive and complicated safety features and measures can be reduced or eliminated. Automatic medicament delivery devices which are easy to operate and low-cost are thereby made possible and can be used conveniently, comfortably and easily by a patient for automatic subcutaneous administration of a preset volume of a liquid medicament at a previously fixed time.

In one embodiment, the present invention comprises a device for the treatment of dawn phenomenon in diabetics, wherein the device is adapted to provide the amount of insulin which is required in the morning at the correct time without the diabetic needing to undertake an injection. When the patient goes to bed, the device according to the present invention may be put on, e.g. by means of a plaster or fastening strap, and a cannula associated therewith can be attached subcutaneously to the body, e.g. by inserting the cannula, designed as infusion needle, into the body.

In some embodiments, a device in accordance with the present invention may have a reservoir which, in an unused state, may be filled with a physiological solvent. The reservoir or solvent may then be supplemented, e.g. before the device is put on, with a required amount of liquid medicament, for example insulin, this being possible, for example, by injecting the liquid medicament through a septum into the reservoir. At a preset time or after a preset delay time, the device then self-actuates to automatically administer a preset amount of medicament without the patient waking up.

In some embodiments, the device according to the present invention may be configured so that it inserts an infusion needle into the body only shortly before conveying of the medicament starts, so that the patient, who is asleep at this time, does not consciously perceive the insertion and the pain associated therewith. After getting up in the morning, the patient removes the device and carries out therapy as usual during the day.

In some preferred embodiments, a device according to the present invention may have a minimal dead volume associated with a transfer system between reservoir and cannula and, in some embodiments, may be worn directly on the injection site on the body.

In a preferred embodiment of the method according to the present invention and of the device according to the present invention (which together may be referred to hereinafter collectively as “the present invention”), the claimed reservoir employed is a disposable pack of a liquid medicament or a refillable container which comprises or contains a liquid medicament. The advantage in the former case is the possibility of ensuring maximum safety and hygiene, because the reservoir is not filled by the user, and the advantage in the second case is that a smaller amount of waste results and costs can be saved. It is also possible in this case to adjust the type and amount of medicament exactly to a patient's requirements.

In a further preferred embodiment of the present invention, the reservoir used is a disposable pack of a physiological solvent into which a desired amount of liquid medicament can be introduced immediately before use. For this purpose, the disposable pack has a septum which can be pierced by the needle of a syringe filled with the medicament for the purpose of injecting the medicament. The disposable pack may contain a physiological saline solution. The advantage of this embodiment is that the type and amount of medicament can be individually adjusted with, at the same time, a high degree of hygiene and safety, and thus a uniform or slow expulsion is possible without elaborate conveying techniques owing to the large specific volume of the dose of medicament to be conveyed.

In some preferred embodiments, the reservoir employed for the present invention is designed as ampoule or bag. Such packs have been thoroughly tested as hygienic medicament reservoirs and moreover allow the medicament contained in them to be conveyed easily, e.g. by a plug forming the base of the ampoule being displaced in the ampoule, or by pressure being exerted on the bag from outside. In some embodiments, ampoules have the advantage over bags in that, because of their stability and shape, they can be connected in a simple manner to a conveying line. In some embodiments, bags have the advantage in that they generate relatively little packing waste and virtually no work against friction must be expended for conveying out of them through application of pressure.

In some preferred embodiments of the present invention, insulin is employed as liquid medicament, because in this case the advantages of the present invention, especially in the treatment of the dawn phenomenon in diabetics, become particularly evident. It may be preferred in this connection for the reservoir used to be provided with an amount of insulin equivalent to 5 to 15 international insulin units, an amount of insulin equivalent to 8 to 12 international insulin units, or about 10 international insulin units, because these are the typical doses of medicament for treating the dawn phenomenon.

Depending on the application or treatment, a normal insulin may be used, conveyed to the infusion cannula within not more than one hour, or a fast-acting insulin analogue may be used, conveyed to the infusion cannula within a period of a plurality of hours. It may be worthwhile, depending on the conveying device used, to use a highly concentrated insulin (e.g. in the commercially available U100 concentration) or an insulin diluted with a solvent, e.g. physiological saline solution, the advantage in the latter case being that accurate dosage is simplified owing to the relatively large specific volume of the insulin dose.

In a further preferred embodiment of the present invention, the liquid medicament is conveyed to the infusion cannula at a substantially constant conveying rate between the start of conveying and the end of conveying, the resulting advantage being, at least when the conveying takes a plurality of hours, that the expulsion of medicament is substantially uniform over this period, this being particularly advantageous with use of fast-acting insulin analogues.

In yet a further preferred embodiment of the present invention, on actuation of the device according to the present invention, e.g. by operating a particular actuation switch by which simultaneously a particular lead time is fixed, or prior to the actuation of the device, e.g. by programming in a particular lead time or the time of day when conveying is desired to start, the delay until the start of conveying may be selected, chosen or set. This facilitates adaptation of the conveying of the medicament to the profile of need.

Irrespective of whether the medicament reservoir is provided in the form of a disposable pack of the medicament, in the form of a refillable, multi-use container or in the form of a disposable pack of a physiological solvent which is to be supplemented with the liquid medicament shortly before use, in some preferred embodiments a device according to the present invention is intended to be used more than once, wherein the reservoir is replaced or refilled each time, reducing cost. For this purpose, the reservoir and conveying device may be separable from one another and likewise reassembled in a simple manner without tools.

If a disposable pack of the medicament or a disposable pack of a physiological solvent which is to be supplemented with the liquid medicament shortly before use is employed as medicament reservoir, other preferred embodiment of the present invention provides for both the conveying device and the reservoir to be configured and used as disposable articles for single use. In these embodiments, they may be designed together as a unit which cannot be separated, at least not without using tools. Embodiments in which disposable packs of the liquid medicament are employed as reservoir thus result in increased safety with at the same time increased convenience of use. However, even if such disposable devices with a reservoir which is initially filled with a physiological solvent, and which are then “loaded” with the medicament, for example by injecting the medicament through a septum into the solvent present in the reservoir, are provided, a high level of safety and comfort results with, at the same time, increased flexibility in relation to the medicament and the amount of medicament.

In some preferred embodiments of the present invention, the delayed initiation of the conveying of the liquid medicament is effected by an electronic control, thus making particularly accurate initiation possible, including initiation at a particular time of day.

In other preferred embodiments of the present invention, the delayed initiation of conveying of the medicament is effected in a purely mechanical way through the device having a purely mechanical initiation device, in some preferred embodiments with hydraulic or boundary friction-related escapement of an initiation movement. The advantage thereof is the possibility of dispensing with electrical energy sources and the possibility of simple, low-cost and constraint proceeding delivery devices which function reliably and are sufficiently precise.

In a further preferred embodiment, the delay may be implemented by a mechanical escapement with pallet and balance wheel similar to a mechanical alarm clock, or by other suitable mechanical arrangements.

In yet another preferred embodiment of the present invention, the delayed initiation of the conveying of the liquid medicament is effected by a chemical reaction taking place, in some preferred embodiments, through the reaction generating the energy for operating an initiation device of the device and/or for the conveying device of the device. Examples of suitable chemical reactions are chemical reactions which start slowly and which finally proceed very exothermically or with a large increase in volume or pressure. Examples are exothermic oxidation reactions in which the reaction rate increases with increasing temperature, or the reaction of two basic components to give a synthetic foam, e.g. polyurethane foam, which is associated with a large increase in volume. Also envisaged in addition is the generation by electrolysis of a gas which is under increasing pressure and by which the conveying of the medicament is initiated and/or brought about.

In a preferred embodiment of the present invention, electrical energy is employed as the exclusive or at least partial actuation energy, in particular in such a way that an electric motor of the conveying device of the device according to the present invention is actuated with or by electrical current, the heating resistance of a bimetallic actuator, an expansion material actuator. In some embodiments, a shape-memory actuator is operated with electrical current. For these purposes, the device has an electrical energy source which may be formed by a disposable battery, a rechargeable battery or other suitable source. An advantage of such embodiments is the resulting possibility of great flexibility in relation to the controllability of the conveying process.

In a further preferred embodiment of the present invention, the actuation energy employed for conveying the liquid medicament is exclusively or at least partially mechanical energy from a mechanical energy accumulator, e.g. a pre-tensioned spring or a compressed gas. Such energy accumulators are extremely simple and reliable and can moreover store relatively large amounts of energy in a small and light format. A corresponding advantage is that this favours the implementation of low-cost and, at the same time, reliable devices according to the present invention.

In yet a further preferred embodiment of the present invention, the actuation energy employed for conveying the liquid medicament is exclusively or at least partially energy from a chemical reaction, e.g. from a chemical reaction which takes place with expansion, pressure build-up and/or heat evolution. In some preferred embodiments, such a chemical reaction may be used for both the initiation and for the provision of the actuation energy for conveying the liquid medicament. Some examples of suitable chemical reactions have already been mentioned, and another is an electrical or chemical ignition of a pyrotechnical propellant, by which the actuation energy for conveying the liquid medicament can be provided.

In yet a further preferred embodiment of the present invention, the actuation energy employed for conveying the liquid medicament is exclusively or at least partially energy from an osmotic pressure build-up.

The previous two embodiments mentioned have the advantage that very low-cost and reliable devices according to the present invention can be manufactured in mass production, making their design and use as disposable articles commercially efficient.

To minimize a fluidic “dead volume” of a device according to the present invention and, thus, the amount of liquid medicament which cannot be used, in some embodiments it is generally preferred to make the connection between reservoir and cannula as short as possible, for example by connecting them together by a very short transfer tubing with a length of a few cm. However, in some preferred embodiments, the device may be designed in such a way that the cannula pierces a septum of the reservoir on actuation of the device or, at the latest, when the conveying of the medicament is initiated, itself forming a reservoir component from the outset. An advantage is that elaborate and potentially fault-prone coupling points are avoided.

Additional aspects of the present invention consist of applying the method according to the present invention or using the device according to the present invention for delivering a liquid medicament, in some preferred embodiments insulin, into a patient's body, which delivery takes place during the early hours of the morning. Advantages of the present invention become particularly evident with this application and use.

Another aspect of the present invention relates to a disposable pack of an insulin or an insulin analogue for use as reservoir for a device according to the present invention, which comprises an amount of insulin or amount of insulin analogue equivalent to 5 to 15, 8 to 12, or 10 international units of insulin.

Another aspect of the present invention relates to using a disposable pack of a physiological solvent, e.g. a physiological saline solution, to form a reservoir for a device according to the present invention, having a septum for delivering a liquid medicament by a syringe, pen or the like into the solvent packed in the disposable pack.

The packs according to the present invention may be commercial products and may be employed both as part of disposable devices according to the present invention and as part of reusable devices according to the present invention.

### BRIEF DESCRIPTION OF THE DRAWINGS

FIGS. 1a and 1b depict idealized time courses of a delivered medicament volume V and conveying rate Q of a device according to the present invention in operation;

FIGS. 2a to 2c schematically depict a first exemplary device according to the present invention before the start, during and after completion of the conveying or delivery of a medicament;

FIGS. 3a and 3b schematically depict a second exemplary device according to the present invention before and at the start of the conveying of medicaments;

FIGS. 4a and 4b schematically depict a third exemplary device according to the present invention before and after the start of the conveying of medicament;

FIGS. 5a and 5b schematically depict a fourth exemplary device according to the present invention before the start and at the end of the conveying of medicament;

FIGS. 6a and 6b schematically depict a fifth exemplary device according to the present invention before the start and after the completion of the conveying of medicament; and

FIGS. 7a and 7b schematically depict a sixth exemplary device according to the present invention before the start and shortly before completion of the conveying of medicament.

### DETAILED DESCRIPTION

With regard to fastening, mounting, attaching or connecting the components of the present invention to form the device as a whole, unless specifically described as otherwise, conventional mechanical fasteners and methods may be used. Other appropriate fastening or attachment methods include adhesives, welding and soldering, the latter particularly with regard to the electrical system of the device, if any. In embodiments with electrical features or components, suitable electrical components and circuitry, wires, chips, boards, microprocessors, inputs, outputs, displays, control components, etc. may be used. Generally, unless otherwise indicated, the materials for making the device of the present invention and/or its components may be selected from appropriate materials such as metal, metallic alloys, ceramics, plastics, etc.

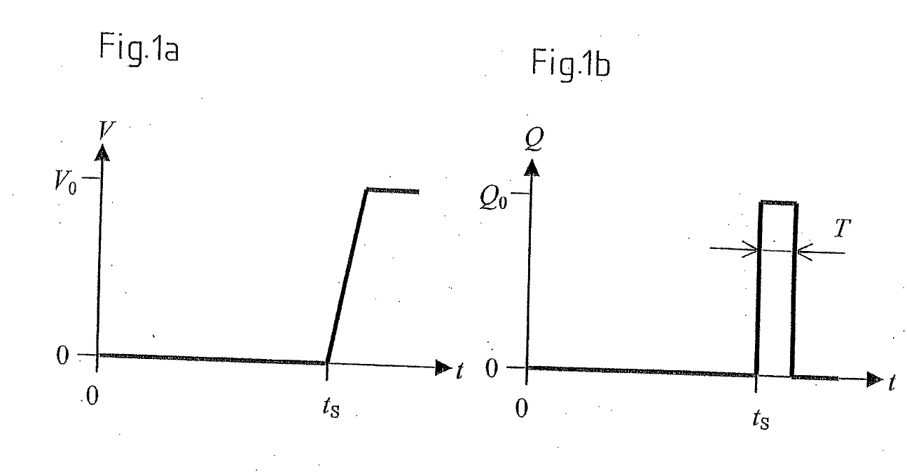

A functional principle of the present invention can be explained in conjunction with FIGS. 1a and 1b which show the idealized time courses of the delivered medicament volume V (FIG. 1a) and of the conveying rate Q (FIG. 1b) of a device according to the present invention on delivery of insulin into a patient's body for the treatment of the so-called “dawn phenomenon”. As is evident, after actuation of the device (t=0) there is initially up to the starting time ts of the conveying only a negligibly small, or no, expulsion of the liquid medicament (V=0, Q=0). Then, starting at ts, an insulin volume V₀ is conveyed over the relatively short time T as shown in FIG. 1a into the patient's body.

The actual courses may differ from the courses shown in FIGS. 1a and 1b depending on the implementation of the device according to the present invention, although in some embodiments the basic course is substantially always the same.

The timing or operation of embodiments of the device and method of the present invention can be divided generally into the following three sections:
1. t<0—putting on and actuation of the device (at time t=0)
2. 0<t<tₛ—delaying
3. tₛ≦t≦tₛ+T—conveying (expulsion)

Both the time of expulsion and the amount of medicament are different among individuals. For expulsion of medicament in the early hours of the morning, e.g. as depicted in FIGS. 1a and 1b, the delay time amounts to some hours. The amount of medicament to be expelled can for example in the case shown be V₀=10 IU, or in other words 10 international insulin units. The duration T of the actual medicament delivery depends primarily on the type of medicament employed. In the case of normal insulin with a total duration of action of a few hours, delivery should take place rather quickly, typically with T<1 hour. If, by contrast, for example a very fast-acting insulin analogue is employed, delivery takes place distinctly more slowly, meaning for example with T=3 hours, for instance. Especially on use of slow-acting medicaments such as normal insulin, both the exact starting time of conveying and the delivery profile are rather uncritical. These variables are more critical with fast-acting medicaments such as, for example, certain insulin analogues.

The figures described below show diagrammatic representations of exemplary preferred embodiments of devices according to the present invention which are suitable, inter alia, for delivering therapeutic substances, including administering insulin delivery as described above for treating the dawn phenomenon in diabetics.

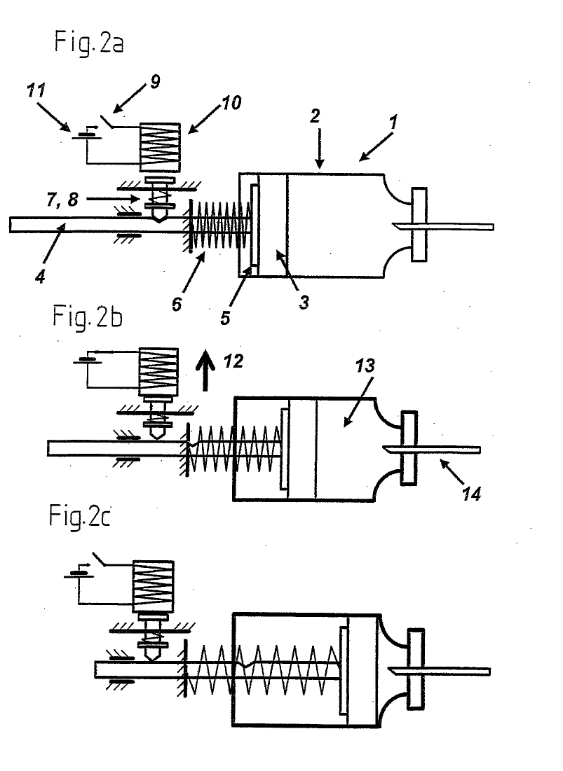

The structure of a first exemplary device according to the present invention is depicted schematically in various states of operation in FIGS. 2a to 2c. This device has a spring actuation with electromagnetic initiation. FIG. 2a shows the device in the resting state before conveying, i.e. at time t<ts. The medicament reservoir 2 consists, in this example, of a cylindrical ampoule 1 with plug 3. The actuation is formed by a piston rod 4 with flange 5 and a compression spring 6. In the depicted state before the conveying of insulin, the spring 6 is tensioned and the piston rod 4 is locked in its position by a locking mechanism consisting of a latch 7 and of a compression spring 8. At time t=ts when conveying starts, an electronic switch 9 is actuated by an electronic control (not depicted), thus applying electric current to an electromagnet 10 by means of an energy source ((rechargeable) battery etc) 11. The retaining latch 7 is thereby moved away from the piston rod in the direction 12 and releases the latter. Detailed technical embodiments of such initiation devices are known in large numbers, e.g. from relay technology and can be applied by the skilled person to this application.

After the piston rod 4 has been released, the actuation spring 6 relaxes, with the medicament 13 being forced by means of flange 5 and plug 3 out of the reservoir 2 and injected through the cannula 14. This situation is depicted in FIG. 2b.

The final state after complete emptying of the reservoir 2 is depicted in FIG. 2c. The tensioning of the actuation spring 6 before the next application takes place for example by pushing the flange 5 down with the aid of a suitable tool or by retracting the piston rod 4.

The duration T of the medicament delivery derives in the present case from the spring force, the friction of the plug of the reservoir 2 and the flow resistance of the cannula 14 (plus that of any transfer system present). Without further measures, T will be in the region of seconds for a typical configuration. Hence the device is suitable in this form for delivering normal insulin. If a delivery time in the region of hours is required for administering insulin analogues, the relaxation of the actuation spring 6 can be delayed deliberately, e.g. by a hydraulic damper. However, in this case, it is advantageous to use the flow resistance of the medicament transfer system itself for damping. To this end, a transfer system attached to the reservoir 2 is formed wholly or partly by a glass capillary through whose length and internal diameter the delivery time T can be adjusted within wide limits.

In one preferred embodiment, the actuation spring 6 can also be a gas compression spring. The delivery time is set, depending on the design of the control, either by setting the delay time between the putting on of the device and the conveying (expulsion) of the medicament or by direct setting of the time of day for expulsion. Instead of the electronically controlled electromagnetic initiation, it is also possible to implement similar systems as already mentioned previously, also with a mechanical escapement, in particular a mechanical escapement with pallet and balance wheel.

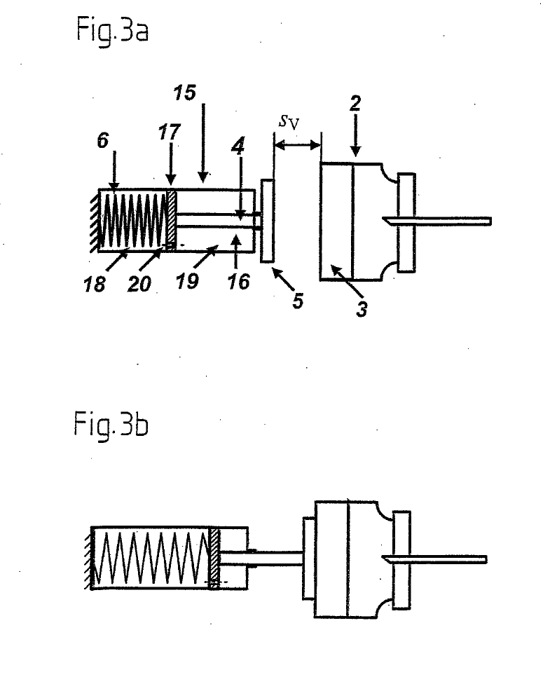

FIGS. 3a and 3b show schematically a second exemplary device according to the present invention with spring actuation, once before the start of conveying (FIG. 3a) and once when conveying starts (FIG. 3b). Since in this embodiment the delaying effect is achieved purely mechanically by a hydraulic damper, no electronic or electromechanical components are required. The actuation spring 6 in this case runs in a damper cylinder 15 which is filled with a high-viscosity liquid 16. The actuation spring 6 operates on a damper piston 17 which runs play-free in the cylinder 15 and divides the latter into two chambers 18 and 19. The single fluidic connection of the two chambers 18, 19 is formed by a bore (or plurality of bores) 20 of small diameter (e.g. 0.1 mm). A piston rod 4 with flange 5 projecting out of the cylinder 15 is firmly connected to the damper piston 17. The pretension of the actuation spring 6 in this case leads to displacement of damper liquid 16 out of chamber 19 into chamber 18 through the bore 20. The high viscosity of the damper liquid 16 and the small diameter of the bore result in a strong damping and a correspondingly slow movement. The delaying effect results from the fact that the flange 5 is initially spaced by a certain delay distance Sv from the plug 3 of the reservoir 2. Conveying of medicament starts only at the time when the flange 5 touches the plug 3. This point in time is depicted in FIG. 3b. A delay time of a plurality of hours can be achieved by suitable design in this way. If the delay distance is designed to be adjustable, it is easily possible for different delay times to be achieved in conjunction with an appropriate adjusting scale. Renewed tensioning of the actuation spring 6 before the next use entails displacement of damper liquid 16 from chamber 18 to chamber 19 and must take place with great force or very slowly. This occurs through defined loading of flange 5 with a weight. Since, in some embodiments, the device is used during the night, a period of a plurality of hours for the tensioning is not critical. It is expedient to provide an appropriate device into which the patient inserts the device after taking it off in the morning and which carries out the tensioning of the spring 6 during the day. However, it is likewise possible to connect the two chambers 18, 19 by a bypass line which has a large cross section and which is blocked during use by a valve or a similar element. After the valve is opened it is then possible, because of the larger flow cross section, for the spring 6 to be tensioned distinctly faster and with less force. The device shown herein is advantageous since it can be implemented in a small size, but other devices are also possible. Thus, for example, it is possible to dispense with filling the chamber 18 with damper liquid and with bore 20. Instead, the chamber 19 is then connected via a capillary (not shown) to a compensating vessel (not shown). Displacement of the damper liquid 16 then takes place from chamber 19 through the capillary into the compensating vessel. Since the capillary is distinctly longer than the bore 20, its diameter can be chosen to be correspondingly larger. As a consequence of the system, the speed of advance of the piston rod 4 in the device shown in FIGS. 3a and 3b falls exponentially over time, so that the delivery stroke must be chosen to be distinctly smaller than the delay stroke so that the expulsion time is not too long. The medicament reservoir 2 should therefore have a large area with a small height. Expulsion time T with a delivery stroke of about 1 mm will typically be in the region of hours.

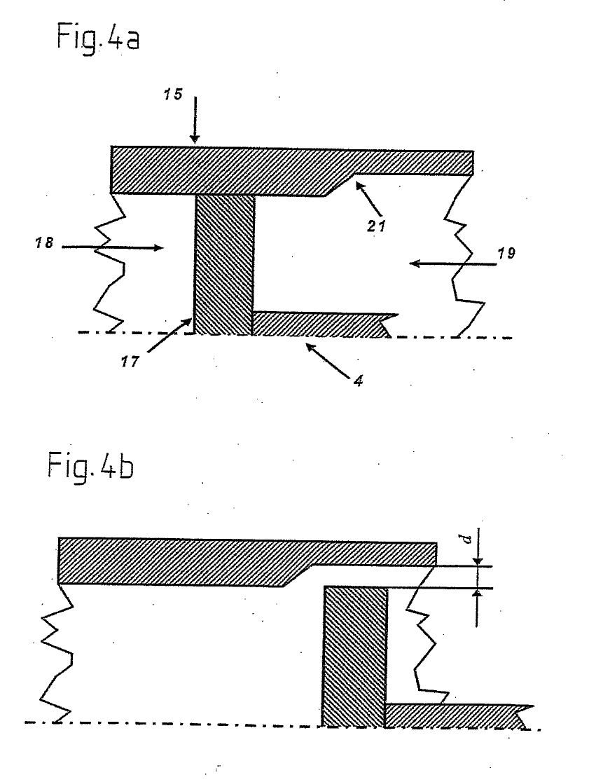

FIGS. 4a and 4b show detailed views of a damper cylinder 15 of a third exemplary device according to the present invention similar to that shown in FIGS. 3a and 3b in section. The difference from the device shown in FIGS. 3a and 3b is, however, that the diameter of the damper cylinder 15 in this case is not constant over its entire length, but shows an (almost) abrupt alteration 21. FIG. 4a shows initially the situation before expulsion, i.e. at a time t<ts. The conditions here correspond to the previous depiction. The initial position of piston rod 4 and damper piston 17 in the damper cylinder 15 is chosen so that the damper piston 17 is located at time t=ts at the alteration point 21 in the cylinder diameter. The larger cylinder diameter for t>ts results in a gap d between cylinder 15 and piston 17, through which gap the damper oil can largely unimpeded flow from chamber 19 to chamber 18, thus greatly reducing or even eliminating the damping. Correspondingly, a rapid expulsion and a larger delivery stroke, which are not straightforwardly achievable with the system shown in FIGS. 3a and 3b, are possible. Also conceivable are embodiments in which a damper device similar to that shown here is employed only to achieve the delay, e.g. by a separate spring being released to actuate the conveying of medicament thereby at time t=ts.

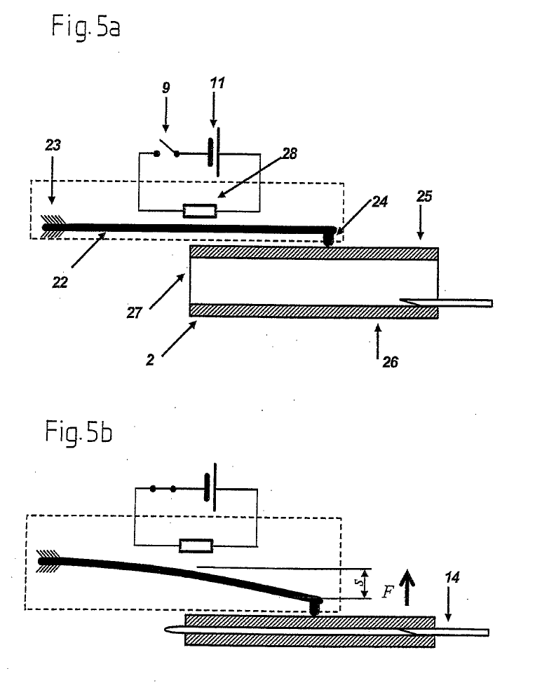

FIGS. 5a and 5b show a fourth exemplary device according to the present invention, once before the start of conveying (FIG. 5a) and once at the end of conveying (FIG. 5b). As is evident, the device shown here has a bimetallic actuator (see broken line) for actuation. This comprises a compound spring 22 of thermo bi-metal which is firmly clamped at one end 23 and is in direct contact with its other end via a thrust piece 24 with the reservoir 2 or shows a small distance (typically <1 mm) therefrom. The reservoir 2 is in this case designed as bag with rigid end areas 25, 26 and flexible side part 27. The reservoir 2 and the bimetallic actuator can in this case be designed either together as (partly sterile) disposable product, or only the reservoir 2 is designed as disposable product, while the bimetallic actuator forms, together with control and energy supply, a reusable basic appliance. In some preferred embodiments, the bimetallic actuator is configured so that the bimetallic spring 22 has a flat shape at t<tₛ. When the thrust piece 24 is already in contact with the reservoir 2, the force exerted by the bimetallic spring 22 on the reservoir 2 is insufficient to displace the end area 25 of the reservoir 2. At time t=tₛ, or shortly beforehand, the bimetallic spring 22 is heated, through closure of an electronic switch 9, by an energy source 11 (FIG. 5b). The heating takes place through an ohmic resistance 28 which is in close thermal contact with the bimetallic spring 22 and can be formed, for example, by a constantan layer applied to the bimetallic spring 22. Alternatively, however, the resistance may also be formed by the bimetallic spring 22 itself. It is also possible to employ for the heating furthermore elements having nonlinear characteristics, in particular a PTC resistor. The heating results in the free end of the bimetallic spring 22 bending outwards in the direction of the reservoir 2, and displacing the end area 25 of the reservoir 2 by the distance s, thus displacing the medicament from the reservoir 2 into the transfer system and into the cannula 14, respectively.

Since a bimetallic compound spring has a relatively small mechanical work capacity, the necessary displacement s and the opposing force F exerted by the reservoir have great importance. Reservoirs having a large base area and a small height and small frictional force are, therefore, advantageous in the present case. These requirements can be satisfied by reservoirs in bag form. A reasonable configuration is possible for instance with s less than or equal to 3 mm and F less than or equal to 3 N. The configuration ought, however, to be such that bending of the bimetallic spring owing to variations in the ambient temperature do not lead to premature expulsion of medicament. This can be achieved either by a certain distance between thrust piece 24 and end area 25 for t<tₛ or by a certain reservoir friction. Instead of a bimetallic compound spring it is also possible to employ a bimetallic snap-action plate which changes its direction of bending virtually instantaneously in a small temperature range on heating. All the statements made hereinbefore in relation to the embodiments with spring actuation apply analogously to the electronic control. Besides the control and the main components shown, the device may comprise further safety and auxiliary units. Mention should be made in this connection of a protection from overheating and a device in the form of a contact, switch etc. for detecting complete emptying of the reservoir, which may be preferred in principle for all embodiments of devices according to the present invention. It is also possible, in just the same way as for the further embodiments described hereinafter with thermal actuators, for the actual delay to be achieved by a mechanical escapement with spring actuation, which actuates the heating by means of an electromechanical switch after the delay time has elapsed.

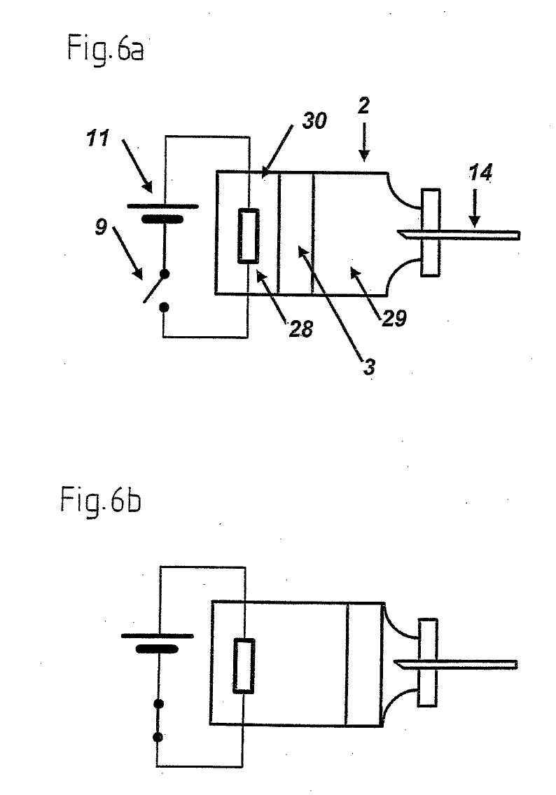

FIGS. 6a and 6b show a fifth exemplary device according to the present invention, once before the start of conveying (FIG. 6a) and once after the end of conveying (FIG. 6b). As is evident, the device shown here has an expansion material actuator with electrical initiation as actuator for conveying the liquid medicament. In this case, the medicament reservoir 2, which is a cylindrical ampoule in the example shown here, is divided into two chambers 29 and 30 which are separated from one another by an axially displaceable plug 3. The medicament is present in the first chamber 29, while the second chamber 30 is filled with a so-called expansion material. Expansion materials mean materials (usually waxes or the like) which have a relatively low melting point and undergo a large increase in volume on melting. FIG. 6a initially depicts the situation t<tₛ when the expansion material is present in the solid state. At time t=tₛ or shortly before, a heating resistance 28 which is surrounded by the expansion material as shown in FIG. 6b is connected to an electrical energy source 11 by closing an electronic switch 9. The expansion material is heated thereby and melts. Owing to the increase in volume associated therewith, the plug 3 is displaced in the axial direction and displaces the medicament out of the first chamber 29 into the transfer system and/or the cannula 14. Instead of a solid expansion material it is also possible to employ liquids such as alcohols having a low specific heat capacity and large thermal expansion. Whereas the expansion material and medicament in the embodiment shown here are present in a joint reservoir, and the expansion actuator is a disposable article, embodiments with a reusable actuator are of course also envisaged. It is likewise possible for the heating resistance 28 optionally also to be arranged outside the expansion chamber 30. It is additionally also possible for the second chamber 30 to be completely filled with an electrolyte solution, instead of an expanding material, and for the displacement of the plug 3 to be carried out by an electrolytic gas generation.

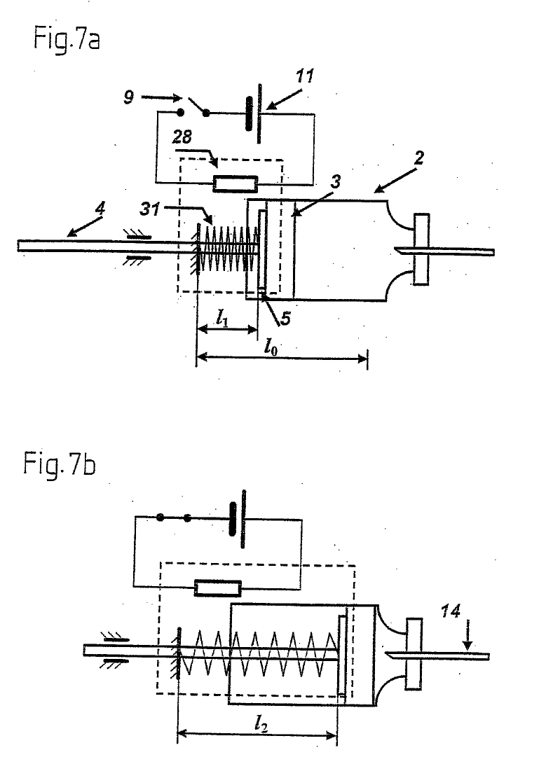

FIGS. 7a and 7b show a sixth exemplary device according to the present invention, once before the start of conveying (FIG. 7a) and once shortly before the end of medicament conveying (FIG. 7b). As is evident, the device shown here has a shape-memory actuator with electrical initiation (see broken line) as actuator for conveying the liquid medicament. The shape-memory actuator comprises a shape-memory element 31 (e.g. on nickel/titanium-basis) which in this case has the shape of a coiled spring of stationary length l₀, a piston rod 4 and a flange 5. FIG. 7a depicts the situation before the start of expulsion, i.e. at a time t<tₛ. The shape-memory element 31 here is compressed to a length l₁ which is less than 10 and touches the plug 3 of the filled medicament reservoir 2. At time t=tₛ or shortly before, the shape-memory actuator is heated by means of an energy source 11 through closure of an electronic switch 9 (FIG. 7b). The heating takes place either by an ohmic resistance 28, which is in close thermal contact with the actuator, or the ohmic resistance of the shape-memory element 31 is used directly for the heating. The heating results in the actuator spring 31 which forms the shape-memory element changing its crystallographic structure in a narrowly limited temperature range from the martensitic to the austenitic state, and it attempts thereby to assume its “imprinted” stationary length l₀. On expansion of the actuator spring 31, the reservoir plug 3 is displaced and the medicament displaced from the reservoir 2 into the transfer system and/or the cannula 14. The configuration is such that the spring length 12 resulting when the reservoir 2 is completely empty is somewhat less than l₀. It is ensured in this way that sufficient force for squeezing out the reservoir 2 is available. Resetting before the next use can take place (after cooling of the shape-memory element 31) by pressing on the flange 5 or retraction of the piston rod 4. Since in this case, in contrast to the spring actuations described previously, only a rather small force is necessary, the pressing of the flange 5 can where appropriate also take place automatically on insertion of the new reservoir 2 by its plug 3. For this the static frictional force of the reservoir plug 3 is more than the restoring force or for a reservoir which is initially closed on the outlet side to be used. The restoring can furthermore take place automatically through an extension spring disposed parallel to the shape-memory element 31. In the present example, the shape-memory actuator is part of a reusable basic appliance. However, it is also envisaged that it be designed as part of the disposable reservoir.

Instead of the electrical resistance heating shown previously for providing heat for the bimetallic, expansion material and shape-memory actuators, it is also envisaged that chemical heat sources could be employed for heating. These can be actuated either immediately on actuation of the device, in order then to initiate a relatively slow exothermic reaction, or else with a delay by a mechanical or electrical control, in order then to generate heat of reaction relatively quickly.

Besides the conveying mechanisms described above, a delayed medicament expulsion can also be achieved by using other energy sources for actuation. Thus, for example, it is also envisaged that conveying of medicament be brought about by decompression at time ts of a previously compressed actuation gas (for example carbon dioxide) and for the medicament to be displaced from its reservoir by expansion occurring thereby. In some embodiments, the gas reservoir employed is a prefilled gas cartridge, in which case the delayed initiation is effected by an electronically or mechanically controlled escapement. It is further envisaged to bring about the conveying of medicament by the electrical ignition of a small pyrotechnical propellant whose explosion is associated with a large expansion. The propellant itself and the igniting electrodes in this case form part of the medicament reservoir and are disposable articles, whereas the electronic control and the device for generating the ignition voltage belong to a reusable basic appliance. Instead of a solid propellant it is also possible to employ an appropriately small quantity of an explosive gas.

Osmotic actuators are also envisaged as further source of actuation energy, where the directed diffusion of a solvent through a semi-permeable membrane is employed to generate a compressive force. It is moreover possible for the osmotic actuator to be employed in the devices according to the present invention in at least two different ways. It may be constructed as osmotic pump which displaces the liquid medicament out of the reservoir through the osmotic pressure, in which case the delay in conveying is achieved by an empty regulating path in a similar way to the device shown in FIGS. 3a and 3b. Or the osmotic pressure which initially builds up slowly after actuation (start of osmotic diffusion) is used for the delay and initiates by a suitable device the expulsion of medicament when a threshold is exceeded, e.g. by releasing a spring actuator which then provides the energy necessary for the conveying.

Embodiments of the present invention, including preferred embodiments, have been presented for the purpose of illustration and description. They are not intended to be exhaustive or to limit the invention to the precise forms and steps disclosed. Obvious modifications or variations are possible in light of the above teachings. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A method for automated delivery of a liquid medicament to an infusion cannula comprising the steps of:
a) providing a reservoir with an amount of the liquid medicament which is non-fatal for a person on short-term subcutaneous administration, and
b) conveying the liquid medicament by an automated conveying device from the reservoir to the infusion cannula, wherein the conveying device is actuated manually and then, with a delay of between four and eight hours, automatically starts conveying the liquid medicament, and wherein, after the start of conveying, the whole amount of the liquid medicament in the reservoir is conveyed to the infusion cannula within a period not exceeding four hours.

2. The method according to claim 1, wherein the reservoir comprises one of a disposable pack of the liquid medicament or a refillable container containing the liquid medicament.

3. The method according to claim 1, wherein the reservoir is provided by introducing a particular amount of the liquid medicament into a disposable pack of a physiological solvent for the liquid medicament.

4. The methods according to claim 3, wherein the physiological solvent is a saline solution, and wherein the liquid medicament is introduced via a septum.

5. The method according to claim 1, wherein the liquid medicament is provided in a reservoir comprising one of an ampoule or bag.

6. The method according to claim 1, wherein the liquid medicament is insulin.

7. The method according to claim 6, wherein the amount of insulin is equivalent to one of 5 to 15 international insulin units, 8 to 12 international insulin units, or 10 international insulin units.

8. The method according to claim 6, wherein a normal insulin is used, and the entire amount of insulin provided is conveyed to the infusion cannula within a period not exceeding one hour.

9. The method according to one of the claim 6, wherein a fast-acting insulin analogue is used, and the complete amount of insulin provided is conveyed to the infusion cannula within a period of from two to four hours.

10. The method according to claim 1, wherein the liquid medicament is conveyed to the infusion cannula at a substantially constant conveying rate between the start and end of conveying.

11. The method according to claim 1, wherein the delay until conveying starts is selected on actuation or prior to the actuation of the conveying device.

12. The method according to claim 1, wherein the conveying device is used more than once, and the reservoir is replaced for each use.

13. The method according to claim 1, wherein the conveying device and the reservoir are used more than once, and the reservoir is refilled for each use.

14. The method according to claim 1, wherein the conveying device and the reservoir are used only once.

15. The method according to claim 1, wherein the conveying of the liquid medicament is initiated by an electronic control.

16. The method according to claim 1, wherein the conveying of the liquid medicament is initiated mechanically.

17. The method according to claim 16, wherein the conveying of the liquid medicament is initiated by hydraulic escapement.

18. The method according to claim 1, wherein the conveying of the liquid medicament is initiated by a chemical reaction.

19. The method according to claim 1, wherein the actuation energy for conveying the liquid medicament is provided at least partly from an electrical energy source.

20. The method according to claim 19, wherein the electrical energy source is a battery.

21. The methods according to claim 19, wherein a motor is actuated by one of the energy source, a bimetallic actuator, an expansion material actuator or a shape-memory actuator.

22. The method according to claim 1, wherein the actuation energy for conveying the liquid medicament is provided at least partly from a mechanical energy accumulator.

23. The method according to claim 22, wherein the accumulator is one of a pre-tensioned spring or a compressed gas.

24. The method according to claim 1, wherein the actuation energy for conveying the liquid medicament is provided at least partly from a chemical reaction that takes place with one of expansion, pressure build-up and heat evolution.

25. The method according to claim 24, wherein the chemical reaction is a pyrotechnical reaction.

26. The method according to claim 1, wherein the actuation energy for conveying the liquid medicament is provided by osmotic pressure build-up.

27. The method according to claim 18, wherein the chemical reaction used to initiate the conveying of the liquid medicament is additionally used to provide the actuation energy for conveying the liquid medicament.

28. The method according to claim 24, wherein the chemical reaction used to initiate the conveying of the liquid medicament is additionally used to provide the actuation energy for conveying the liquid medicament.

29. The method according to claim 1, wherein the liquid medicament is insulin, and is delivered into a patient's body during the early hours of the morning.

30. A device for automated delivery of a substance into a patient's body, comprising:
a cannula for introduction of the substance into the body;
a reservoir containing the substance; and
a conveying device for conveying the substance from the reservoir to the cannula, wherein, after actuation and a delay, the conveying device automatically starts conveying the substance and, after the start of conveying, conveys substantially all the substance to the cannula within a selected time.

31. The device according to claim 30, wherein the actuation is manual, the delay is between four and eight hours, and the selected time does not exceed four hours.

32. A device for automated delivery of a liquid medicament into a patient's body, comprising an infusion cannula for subcutaneous introduction of the medicament into the body, a reservoir containing one of an amount of the liquid medicament which is non-fatal for a person on short-duration subcutaneous administration, and an automated conveying device for conveying the liquid medicament from the reservoir to the infusion cannula, wherein, after actuation and a delay of from four to eight hours the conveying device automatically starts conveying the liquid medicament and, after the start of conveying, conveys the complete amount of liquid medicament in the reservoir to the infusion cannula within a period not exceeding four hours.

33. The device according to claim 32, wherein the reservoir comprises one of a disposable pack of the liquid medicament or a refillable container.

34. The device according to claim 32, wherein the reservoir comprises a disposable pack of a physiological solvent for the liquid medicament

35. The device according to claim 34, wherein the solvent is a saline solution.

36. The device according to claim 32, wherein the reservoir has a septum for injecting a liquid medicament into its interior.

37. The device according to claim 32, wherein the reservoir comprises one of an ampoule or a bag.

38. The device according to claim 32, wherein the liquid medicament is insulin.

39. The device according to claim 38, wherein the reservoir contains the equivalent of one of 5 to 15 international insulin units, 8 to 12 international insulin units, and 10 international insulin units.

40. The device according to claim 39, wherein the insulin is a normal insulin, and the conveying device conveys the complete amount of insulin in a period not exceeding one hour.

41. The device according to claim 39, wherein the insulin is a fast-acting insulin analogue, and the conveying device conveys the complete amount of insulin in a period of from two to four hours.

42. The device according to claim 32, wherein the conveying device conveys the liquid medicament at a substantially constant conveying rate between the start and end of conveying.

43. The device according to claim 32, wherein the conveying device comprises an input for selecting or adjusting the delay.

44. The device according to claim 32, wherein the conveying device and the reservoir are separable.

45. The device according to claim 32, wherein the conveying device and the reservoir are inseparable.

46. The device according to claim 32, further comprising an electronic control for initiating conveying the liquid medicament.

47. The device according to claim 32 further comprising a mechanical initiation device for initiating conveying the liquid medicament.

48. The device according to claim 47, wherein the initiation device involves hydraulic escapement.

49. The device according to claim 32, further comprising an initiation device initiates conveying of the liquid medicament as a result of a chemical reaction.

50. The device according to claim 32, further comprising an energy source for at least partial provision of the actuation energy for conveying the liquid medicament, and a motor actuable by the energy source.

51. The devicee according to claim 50, wherein the energy source is one of a battery, a bimetallic actuator, an expansion material actuator or a shape-memory actuator.

52. The device according to claim 32, further comprising a mechanical energy accumulator for providing at least part of the actuation energy for conveying the liquid medicament.

53. The device according to claim 52, wherein the energy accumulator is one of a pre-tensioned spring or a container filled with compressed gas.

54. The device according to claim 32, wherein the actuation energy for conveying the liquid medicament is at least partly provided by a chemical reaction.

55. The device according to claim 54, wherein the chemical reaction takes place with at least one of expansion, pressure build-up or heat evolution.

56. The device according to claim 54, wherein the chemical reaction involves a pyrotechnical propellant.

57. The device according to claim 32, wherein an osmotic actuation provides the actuation energy for conveying the liquid medicament.

58. The device according to claim 54, further a reaction chamber in which the chemical reaction is started on actuation of the device, wherein the reaction, after a delay, provides the actuation energy for conveying the liquid medicament.

59. The device according to claim 32, wherein the liquid medicament is insulin and is delivered into a patient's body during the early hours of the morning.

60. A disposable pack of insulin for use as reservoir for a device for automated delivery of a liquid medicament into a patient's body, the pack comprising the equivalent of one of 5 to 15, in particular 8 to 12 and in particular 10 international units of insulin.

61. A disposable pack of a physiological solvent for use as reservoir for a device for automated delivery of a liquid medicament into a patient's body, the pack comprising a septum for enabling the delivery of a liquid medicament by a syringe into the solvent in the pack.

62. The disposable pack according to claim 45, wherein the solvent is a saline solution.
